(19) United States
(12) Patent Application Publication
(10) Pub. No.: US 2023/0369033 A1
(43) Pub. Date: Nov. 16, 2023

(54) Methods and Systems for Feedback Control in Plasma Processing Using Radical Sensing

(71) Applicant: MKS Instruments, Inc., Andover, MA (US)

(72) Inventors: Keith K. Koai, Andover, MA (US); Chenglong Yang, Andover, MA (US); Guy Rosenzweig, Andover, MA (US); Jimmy Liu, Andover, MA (US); Michael Harris, Andover, MA (US); James Blessing, Andover, MA (US)

(21) Appl. No.: 17/983,934

(22) Filed: Nov. 9, 2022

Related U.S. Application Data
(60) Provisional application No. 63/278,837, filed on Nov. 12, 2021.

(51) Int. Cl. H01J 37/32; G01N 33/00

(52) U.S. Cl. CPC H01J 37/3299; G01N 33/0027; H01J 37/32449; H01J 2237/24585; H01J 37/32357

(57) ABSTRACT

An apparatus for feedback control in plasma processing systems using radical sensing, and a method for feedback control in plasma processing systems using radical sensing, the apparatus comprising at least one process gas supply system configured to output at least one process gas, at least one plasma source configured to receive the at least one process gas and generate at least one radical flow, at least one process chamber in communication with the at least one plasma source, wherein the process chamber receives the at least one radical flow and directs at least a portion of the at least one radical flow to one or more devices, the process chamber configured to output at least one process chamber output, at least one gas analyzer in communication with and configured to sample at least one of the at least one process gas, at least one radical flow, at least one radical flow within the at least one process chamber, and the at least one process chamber output, and at least one controller in communication with at least one of the process gas supply system, at least one plasma source, and at least one process chamber, the controller configured to generate at least one control signal based on data from the at least one gas analyzer and selectively control at least one of the process gas supply system, at least one plasma source, and at least one process chamber.

### CROSS-REFERENCE TO RELATED APPLICATIONS

This patent application claims the benefit of U.S. Prov. Ser. No. 63/278,837 filed on Nov. 12, 2021, application that is incorporated by reference herein.

### BACKGROUND OF THE INVENTION

### Field of the Invention

This invention relates to system for feedback control and to a method for feedback control. More specifically, this invention pertains to a method and system for feedback control in plasma processing using radical sensing.

### Description of Related Art

Direct plasma processing systems (e.g. Capacitively Coupled Plasma (CCP)/Inductive Coupled Plasma (ICP) and remote plasma sources are frequently used to modify or otherwise treat surfaces during various semiconductor manufacturing operations, flat-panel display manufacturing operations, and the like. For example, plasmas may be employed during etching processes to aid in the formation of complex electrical components and circuits on a workpiece. In addition, plasma processing is used to deposit materials on the surface of a semiconductor wafer.

Typically, wafer processing requires the generation of a consistent concentration of radicals, over extended processing times. Known systems are unable to measure radical concentration in-situ and, as such, these known systems rely instead on an estimated radical concentration, estimation that is based on various operational parameters, or rely on an iterative correction process to achieve a desired radical concentration, either one of which are deterministic to the reaction end result. These methods have a number of shortcomings. For example, the desired radical concentration may be achieved only after a laborious trial-and error correction effort using the wafer process result and off-line metrology as confirmation tool. This practice inevitably is very expensive and interruptive to the manufacturing process. In addition, the radical yield often drifts over time due to any number of factors, including cold start events, aging of components in the power supply or transport system, changes in surface conditions in the transport system or processing chamber, and the like. FIG. 1 shows graphically a typical transient behavior of the concentration of radicals from a radical source during a cold start. As shown, the concentration of radicals being supplied varies from over two hundred parts per million (200 ppm) to less than about seventy five parts per million (75 ppm). As a result, the wafers being processed using such an unstable radical concentration may not prove to be usable wafers, thereby reducing processing yield.

In light of the foregoing, there is a need for an in-situ method and system for feedback control in plasma processing using radical sensing or a control architecture for wafer processing applications that uses radical sensing to control the plasma process.

### BRIEF SUMMARY OF THE INVENTION

The present invention has been conceived and developed aiming to provide solutions to the above stated objective technical needs, as it will be evidenced in the following description.

In accordance with an embodiment of the present invention is proposed an apparatus for feedback control in plasma processing systems using radical sensing, comprising at least one process gas supply system configured to output at least one process gas, at least one plasma source configured to receive the at least one process gas and generate at least one radical flow, at least one process chamber in communication with the at least one plasma source, wherein the process chamber receives the at least one radical flow and directs at least a portion of the at least one radical flow to one or more devices, the process chamber configured to output at least one process chamber output, at least one gas analyzer in communication with and configured to sample at least one of the at least one process gas, at least one radical flow, at least one radical flow within the at least one process chamber, and the at least one process chamber output, and at least one controller in communication with at least one of the process gas supply system, at least one plasma source, and at least one process chamber, the controller configured to generate at least one control signal based on data from the at least one gas analyzer and selectively control at least one of the process gas supply system, at least one plasma source, and at least one process chamber.

In accordance with further aspects of the present invention, during use of the apparatus the at least one plasma source is configured to generate low-energy ions and atomic radicals in the at least one radical flow, directed into one or more process chambers. The at least one process chamber is configured to have one or more substrates or devices positioned therein to be plasma processed. The at least one gas analyzer further comprises at least one mass spectrometer. Exemplarily, the mass spectrometer is a residual gas analyzer, such as the RGA. The at least one controller comprises a mass flow controller, and another flow control device. The at least one controller, during use, is capable of continuously adjusting one or more operational parameters of the apparatus based on gas analyzer data received from the at least one gas analyzer sampling, in real-time, the radical gas flow from said plasma source.

In accordance with an embodiment of the present invention is also proposed a method, comprising selecting a reactor configuration, determining a radical sensing unit to be employed, determining a reaction rate target, setting a radical concentration target, determining a preset flow of other reactants, flowing one or more process gases into at least one plasma source by initiating at least one plasma reaction, measuring a radical concentration using the radical sensing unit, and measuring a reaction rate.

In accordance with further aspects of the present invention, selecting a reactor configuration comprises selecting of any one or a combination of a plurality of processing gases to be used by the reactor, a plurality of materials to be applied on the reactor, a wafer size to be housed by the reactor, a plurality of dimensions for the reactor, and a type of a remote plasma source for the reactor. The radical sensing unit comprises at least one of a mass spectrometry system, and a special residual gas analyzer. Exemplarily, the mass spectrometry system may be an RGA-like special mass spectrometer. The reaction rate targets comprise a target deposition rate, a target process time, an etch rate, and a surface modification treatment rate. Setting a radical concentration target is impacted by at least an initial dose of any gases included in a plasma chamber, and the dose is dependent upon at least one of disassociation rate by pressure, flow rate, power, and thermal management variables. The process gases comprise at least one of O2, N2, H2, NH3, NF3, F2, Cl2, AsH3, BCl3. Br2, CF4, C2F6, C3F8, C4F8, C5F8, CHF3, HBr HCl, HF, N2O, PH3, SiF4, SiH4, SF6. Exemplarily metal inorganic precursors may be TiCl4, WF6. The reaction rate may be measured by at least a laser interferometer capable of examining an etch rate deposition rate. The method may further comprise the step of optimizing a performance of an apparatus for the feedback control by repeating the steps of the method. The method may further comprise the step of adjusting at least one of or a combination of a gas flow rate, power, cooling characteristics prior to providing the gas for plasma reaction. The method may further comprise the step of adjusting at least one of or a combination of a flow rate, and gas mix ration prior to providing the precursor gas for plasma reaction.

More detailed explanations regarding these and other aspects and advantages of the invention are provided herewith in connection with the exemplary embodiments of the present invention.

### BRIEF DESCRIPTION OF THE SEVERAL VIEWS OF THE DRAWING(S)

The above and other aspects, features and advantages of the present invention will become more apparent from the subsequent description thereof, presented in conjunction with the following drawings, wherein:

FIG. 1 is a graphical illustration of a transient behavior of the concentration of radicals from a radical source during a cold start;

FIG. 2 is a block diagram of an embodiment of a method for utilizing feedback control to measure a radical concentration within a gas flow, to control a plasma processing operation;

FIG. 3 is a schematic representation of an apparatus having a radical concentration feedback architecture, in accordance with one embodiment of the present invention.

FIGS. 4-6 illustrate graphs showing that a target flow of a nitrogen radical may be controlled in various ways envisioned in accordance with various embodiments of the present invention.

FIG. 7 is another schematic representation of an apparatus having a radical concentration feedback architecture, in accordance with another embodiment of the present invention.

FIG. 8 is yet another schematic representation of an apparatus having a radical concentration feedback architecture, in accordance with yet another embodiment of the present invention.

FIGS. 9-11 illustrate graphs showing that effective feedback control for the radical concentration may be achieved by controlling the plasma pressure, in accordance with yet another embodiment of the present invention.

FIG. 12 is a further yet schematic representation of an apparatus having a radical concentration feedback architecture, in accordance with yet another embodiment of the present invention.

FIGS. 13-15 illustrate graphs showing that effective feedback control of radical concentration may be achieved by controlling the plasma power, in accordance with yet another embodiment of the present invention.

FIG. 16 shows yet another alternate embodiment of a processing apparatus having a radical concentration feedback architecture.

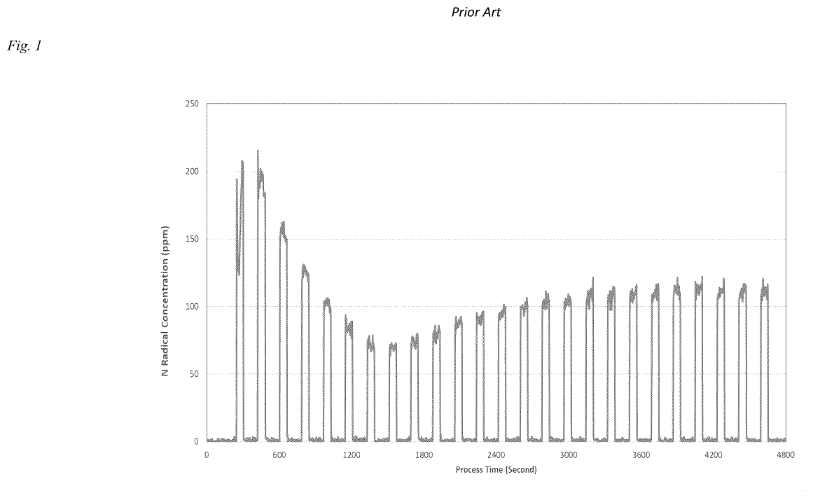

### DETAILED DESCRIPTION OF THE INVENTION

The following description of the presently contemplated best mode of practicing the invention is not to be taken in a limiting sense, but is made merely for the purpose of describing the general principles of the invention. The scope of the invention should be determined with reference to the claims.

The present application discloses various embodiments of methods and systems for feedback control for use in plasma processing using radical sensing. Exemplary embodiments are described below with reference to the accompanying drawings. Unless otherwise expressly stated, in the drawings the sizes, positions, etc., of components, features, elements, etc., as well as any distances therebetween, are not necessarily to scale, and may be disproportionate and/or exaggerated for clarity.

The terminology used herein is for the purpose of describing particular example embodiments only and is not intended to be limiting. As used herein, the singular forms “a,” “an” and “the” are intended to include the plural forms as well, unless the context clearly indicates otherwise. It should be recognized that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise specified, a range of values, when recited, includes both the upper and lower limits of the range, as well as any sub-ranges therebetween. Unless indicated otherwise, terms such as “first,” “second,” etc., are only used to distinguish one element from another.

Unless indicated otherwise, the term “about,” “thereabout,” etc., means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art.

Many of the embodiments described in the following description share common components, device, and/or elements. Like named components and elements refer to like named elements throughout. For example, the embodiments described in the following detailed description generally include at least one processing gas supply, at least one additional reactant supply, at least one remote plasma source or similar plasma source, at least one mass spectrometer, and at least one controller, although those skilled in the art will appreciate that any variety of additional devices or components may be used in the embodiments described below. Thus, the same or similar named components or features may be described with reference to other drawings even if they are neither mentioned nor described in the corresponding drawing. Also, even elements that are not denoted by reference numbers may be described with reference to other drawings.

Many different forms and embodiments are possible without deviating from the spirit and teachings of this disclosure and so this disclosure should not be construed as limited to the example embodiments set forth herein. Rather, these example embodiments are provided so that this disclosure will be thorough and complete, and will convey the scope of the disclosure to those skilled in the art.

Reference will now be made to the drawings wherein like numerals refer to like parts throughout.

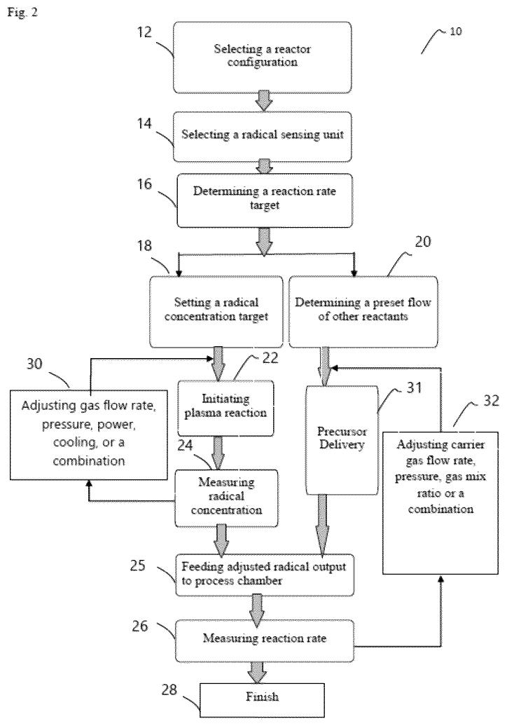

FIG. 2 shows a block diagram of an embodiment of a method for utilizing feedback control to measure a radical concentration within a gas flow, to control a plasma processing operation. As shown in FIG. 2, the method 10 includes at least the step of selecting a reactor configuration 12. Considerations pertinent to selecting the reactor configuration 12 may include the selection of any of parameters including any one or a combination of a plurality of processing gases to be used by the reactor, a plurality of materials to be applied on the reactor, a wafer size to be housed by the reactor, a plurality of dimensions for the reactor, a type of in-situ plasma source, and a type of a remote plasma source for said reactor, or further such reactor specific considerations. Selecting the reactor configuration is corelated with the process step needs, e.g. in plasma etch, what is needed are either isotropic radical etch or anisotropic ion & radical etch. Thereafter, a radical sensing unit to be employed is determined at step 14. Factors relied upon for this determination are at least the radical chemical species, and the intrinsic property of the energy bands. Exemplary radical sensing units include, for example, mass spectrometry systems, special residual gas analyzers, and the like. Exemplarily, the radical sensing unit may be an RGA-like special mass spectrometer. In one particular embodiment, the radical sensing unit may comprise from a modification of the Microvision 2 residual gas analyzer, manufactured by MKS Instruments, Inc. Those skilled in the art will appreciate that a variety of alternative devices may also be used. Thereafter, a reaction rate target may be determined at step 16. Factors relied upon for this determination are at least the process step needs, such as the need of a layer of 200 nm ILD SiOCN layer within 100 sec deposition time. Exemplary such reaction rate targets include, without limitation, the value targets of the deposition rate, of the process time, of the throughput, and the like. The rate is depending upon the process, such as 1 nm/cycle like ALD, or 2-10 nm/s like CVD. Further, a radical concentration target may be set at step 18. Those skilled in the art will appreciate that any number of variables influence the setting of the radical concentration target at step 18. For example, the initial dose of any variety of gases included is one of such variables, which in turn may be based on number of factors such as disassociation rate by pressure, flow rate, power, and thermal management considerations. Further, one or more other reactants may be used. As such, for the illustrated embodiment of method 10, a preset flow of other reactants, consisting at least of either precursor or carrier gases, may be determined at step 20. Both liquid and solid precursors are set at a concentration target by its evaporation pressure and carrier (Ar) gas flow. Selection of inorganic or organic precursor to deliver the species (normally metal) depends on chamber design. For instance, to create a TiN film by CVD with TiCl4/NH3 process is expensive and very corrosive, whereas applying the TDMAT/NH3 process at lower temperature is less expensive. Subsequent plasma treatment might be needed to eliminate carbon, hydrogen, and oxygen.

Referring to FIG. 2, at least one plasma reaction may be initiated at step 22, to generate plasma radicals by flowing one or more process gases into at least one plasma source. A variety of process gases may be used, including, without limitation, O2, N2, H2, NH3, NF3, F2, Cl2, AsH3, BCl3. Br2, CF4, C2F6, C3F8, C4F8, C5F8, CHF3, HBR, HCl, HF, N2O, PH3, SiF4, SiH4, SF6, TiCl4, WF6, and the like. Also, a variety of precursor or carrier gases may be used, that include, without limitation, TMA, CCTBA, HfCl4+H2O, TMDA+O3, SiH4, Si2H6, PDMAT, WF6/TMA, SiH2Cl6, GeH4, NH3, TEOS, DMDMOS, W(CO)6 carbonyl, CH3COCH3, CH3OH, C2H5OH, (CH3)2CHOH, CH3O(CH2)3OOCCH3, C2H5OOCCC(OH)CH3, C4H6ON(CH3O(NMP), C4H8SO2, CH3(CO)C5H11 (2-Heptanone), NH9Si(CH3)3)2 (HMDS), Si(OC2H5)4 (TEOS), PO(0C2H5)4 (TEPO), and the like. Thereafter, the radical concentration may be measured at step 24 using the radical sensing unit determined as discussed above in step 14. The systems and methods described herein enable in-situ, real-time measurement of reactive radical concentration within the gas flow within the plasma chamber. The measured radical concentration of step 24 measured by the radical sensing unit of step 14 may be used to adjust at least one characteristic of the plasma formed during the plasma reaction step 22. The adjustment takes place during a method step 30, and consists at least of comparing setpoints and adjust power, flow rate, pressure, cooling or a combination of these factors to achieve target radical output. Continuous feedback regarding these factors is fed to the actuator as a command, until the target is met. For example, the characteristics of the plasma may be adjusted insofar a number of characteristics associated with the flow of at least one process gas, including adjusting gas flow rate, pressure, power, thermal characteristics, and the like. The precursor determined at step 20 is delivered to the process chamber at step 31. The adjusted radical output is then fed into process chamber at step 25, and a reaction happens on wafer surface with the preset precursor or other reactant that has been delivered at step 31 from the precursor delivery system. An on-wafer reaction may take place in the process chamber at step 25. In addition, the reaction rate may be measured at a subsequent step 26 and, in response, at least one characteristic of the flow of one or more additional reactants or precursors may be similarly adjusted, as it will be discussed further in connection with step 32. In one subsequent embodiment, the reaction rate may be measured at step 26 using for example a laser interferometer to examine etch rate, deposition rate, and the like. Those skilled in the art will appreciate that a variety of alternative systems may also be used to measure the reaction rate. Optionally, steps 12 through 26 may be repeated to optimize the performance of the system, and/or to permit unit-to-unit matching or process chamber matching. At a step 28 the sequence of steps of the feedback control method is finished and this sequence of steps is repeated for the next wafer, unit-to-unit or chamber matching.

Should the measurement of the radical concentration yield results, at step 24, that need to be further optimized, the gas flow rate, power, cooling characteristics, or all of these parameters may be further adjusted at step 30, prior to providing the gas for plasma reaction at step 22. Should the measurement of the reaction rate yield results, at step 26, that need to be further optimized, flow rate, gas mix ration or all of these parameters may be further adjusted at step 32, prior to providing the precursor gas for plasma reaction at step 22. At step 32 the adjustment is made on flow and pressure control, such as carrier gas flow rate, pressure, gas mix ratio or a combination of these is adjusted to achieve the desired reaction rate.

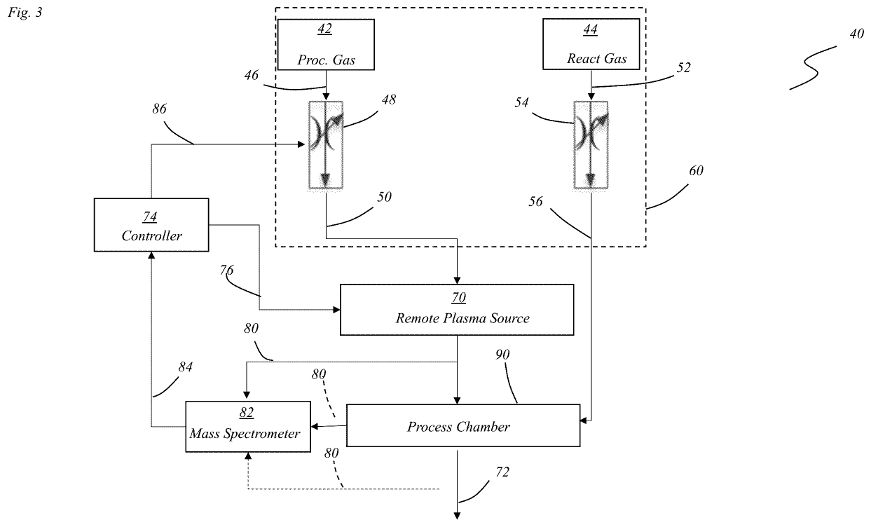

FIG. 3 is a schematic representation of an apparatus having a radical concentration feedback architecture, in accordance with one embodiment of the present invention. As shown, the apparatus 40 may include at least one process gas supply system 42 and at least one additional reactant or precursor supply 44. Optionally, those skilled in the art will appreciate that the system 40 need not include an additional gas reactant supply 44. The process gas supply system 42 outputs at least one gas flow 46 into one or more flow control devices 48. The flow control device 48 may comprise at least one controller (such as a mass flow controller, MFC), although those skilled in the art will appreciate that other flow control devices may be used in the present apparatus. The flow control device 48 is configured to output at least one process gas flow 50 which may be directed into at least one plasma source 70. The plasma source 70 may comprise at least one remote plasma source, although those skilled in the art will appreciate that a variety of plasma sources or plasma generating devices may be used. In one specific embodiment for apparatus 40, the plasma source 70 comprises a Paragon remote plasma source, manufactured by MKS Instruments, Inc. Optionally, the plasma source 70 may comprise one or more CCP sources, ICP sources, or other direct plasma sources known in the art. During use, the plasma source 70 is configured to generate low-energy ions and atomic radicals in a radical flow 72 which may be directed into one or more process chambers 90. The additional reactant supply 44 is configured to output at least one precursor gas flow 52 that may be introduced into the radical flow 72 emitted from the plasma source 70. In the illustrated embodiment, at least one flow control device 54 may be used to control the flow of the additional reactant gases 52 from the additional reactant supply 44 which may be configured to bypass the plasma source 70. As shown, the additional reactant gases 56 from the flow control device 54 may be directed into at least one process chamber 90.

Referring again to FIG. 3, the mass spectrometer (that exemplarily is at least one gas analyzer (RGA)) 82 may be configured to sample at least a portion of the radical flow 72 prior to the radical flow 72 entering the process chamber 90. In the illustrated embodiment, the mass spectrometer 82 comprises at least one mass spectrometer, although those skilled in the art will appreciate that a variety or number of gas analyzers may be used with the present apparatus. Optionally, the mass spectrometer 82 may be configured to receive at least one gas analyzer sample 80 from the process chamber 90 and/or the process chamber output. As shown, a gas analyzer sample 80 is analyzed by the mass spectrometer 82 which in turn generates gas analyzer data 84 which may be provided to one or more controllers 74. In the illustrated embodiment, the controller 74 may be configured to generate one or more control signals 84, 76 which may be provided to at least one of the flow control device 48 and the plasma source 70. Optionally, the controller 74 may provide control signals to any number of components or subsystems in the apparatus 40. For example, controller 74 may be in communication with and control the process gas supply system 42, the additional reactant supply 44, and/or the flow control device 54. Further, the controller 74 may be in communication with one or more external networks, controllers, or control systems. During use, controller 74 may continuously adjust one or more operational parameters of the system 40 based on real-time gas analyzer data 84 received from the mass spectrometer 82 which is sampling, in real-time, the radical gas flow 72 from the plasma source 70.

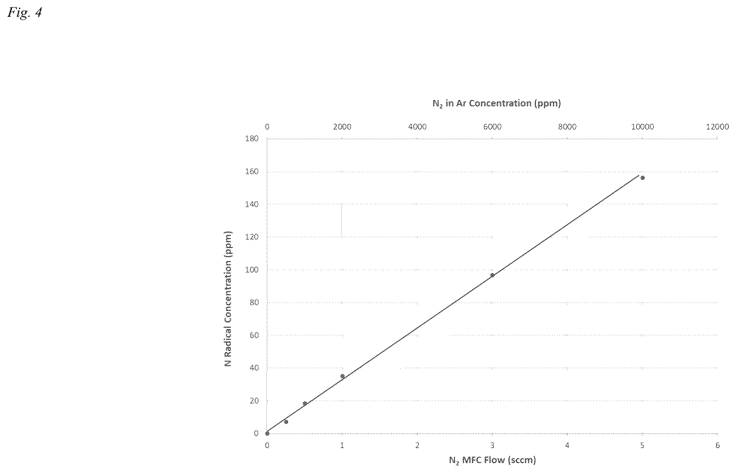

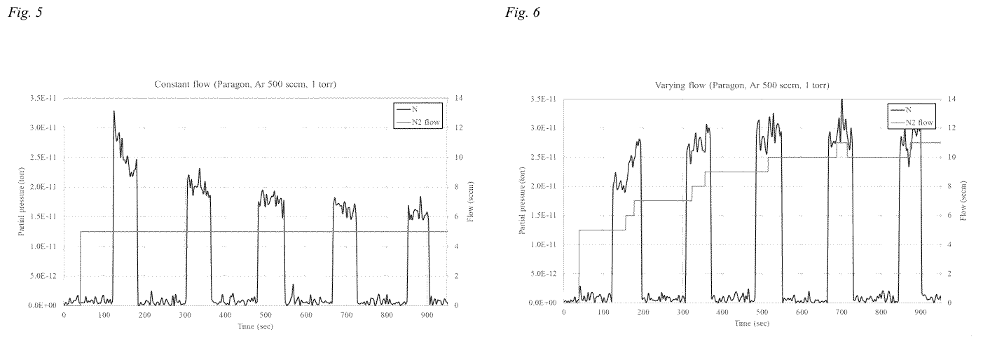

FIGS. 4-6 illustrate graphs showing that a target flow of a nitrogen radical may be controlled in various ways envisioned in accordance with various embodiments of the present invention. More specifically, as shown in FIG. 4, the nitrogen radical concentration from the plasma source 70 may be controllably adjusted either by (a) the flow of N2 into the plasma source 70; or (b) increasing the N2 concentration in the additional reactant flow 50 (e.g. Ar); or increasing the flow of Ar and N2, while keeping the same mixing ratio. FIGS. 5 and 6 show the effects of increasing the flow of nitrogen process gas 50 into the plasma source 70 on nitrogen radical concentration in the radical gas flow 72 stemming out of the plasma source 70. As shown in FIG. 5, the nitrogen radical concentration of the radical gas flow 72 from the plasma source 70 will decrease over time if the flow of process gas 46 from the process gas supply system 42 remains constant. In contrast, as shown in FIG. 6, the nitrogen radical concentration of the radical gas flow 72 from the plasma source 70 will increase over time as the flow of process gas 46 from the process gas supply system 42 is increased. While FIGS. 4-6 show the effects of changing flow rate and concentrations of nitrogen with respect to argon, it has been observed that changing flow rate and concentrations of other process gases would result in a similar increase in the radical concentration in the plasma source output 72.

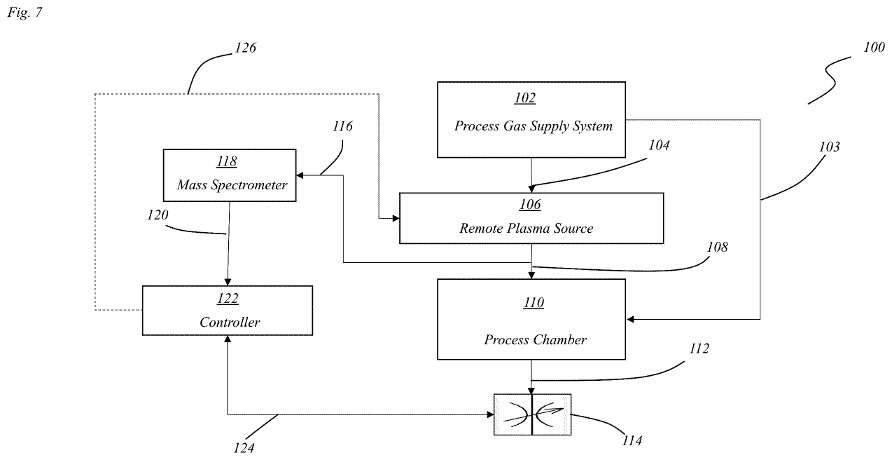

FIG. 7 is another schematic representation of an apparatus having a radical concentration feedback architecture, in accordance with another embodiment of the present invention. As shown, the apparatus 100 includes at least one process gas supply system 102. In the illustrated embodiment, the process gas supply system 102 is comparable to the process gas supply system 60 described above and shown in FIG. 3. As shown, the process gas system 60 may generate at least one process gas flow 104 which may be directed to at least one plasma source 106. Like discussed in connection with the previous embodiment, the plasma source 106 is configured to generate at least one radical flow 108 which may be directed to at least one process chamber 110. In addition, the process gas supply system 102 may be configured to output at least one additional reactant flow 103 which may be directed into the process chamber 110. The process chamber 110 is configured to have one or more substrates or devices positioned therein to be plasma processed. The process chamber 110 may be configured to output at least one processing chamber outputs or flows 112 therefrom. Further, at least one valve 114 may be used to control the flow of the output flow 112 from the processing chamber 110. The process chamber 110 may be controlled by or influenced by at least one valve 114 position proximate to or in fluid communication with the output 112 of the process chamber 110. In the illustrated embodiment, the valve 114 comprises a throttle valve, although those skilled in the art will appreciate that any variety or number of valves or flow control devices may be used in the present system.

Referring again to FIG. 7, at least one gas analyzer sample 116 may be extracted from the radical flow 108 and directed to the mass spectrometer 118. Like the previous embodiment, the mass spectrometer 118 may be configured to generate at least one gas analyzer signal 120 which may be directed to at least one controller 122. As shown, the controller 122 may be in communication with the valve 114. During use, the controller 122 may receive position data or flow data from the valve 114 and may send control signals 124 to the valve 114 based on the gas analyzer data 120 received from the mass spectrometer 118. In addition, the controller 122 may be configured to send control signals 126 to the plasma source 106 based on data received from the mass spectrometer 118.

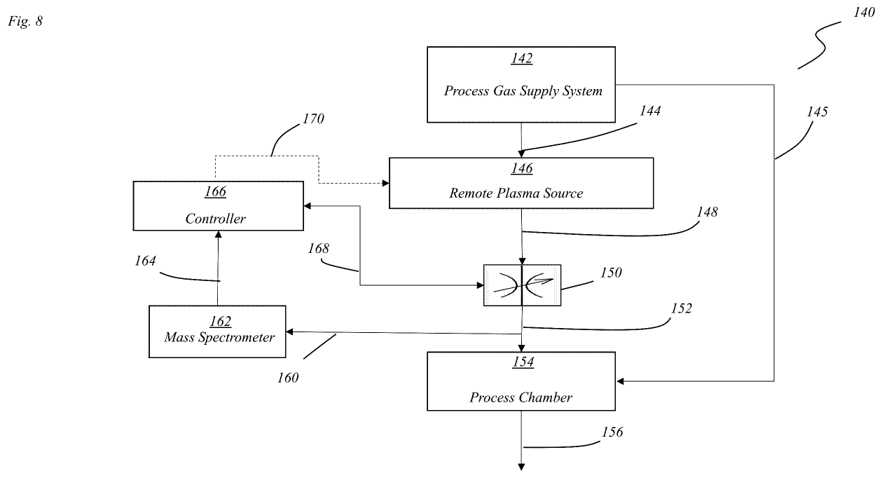

FIG. 8 is yet another schematic representation of an apparatus having a radical concentration feedback architecture, in accordance with yet another embodiment of the present invention. As shown, the apparatus 140 includes at least one process gas supply system 142, similar to the process gas supply systems described above. The process gas supply system 142 may be configured to output at least one process gas flow 144 to at least one plasma source 146 which in turn outputs at least one radical gas flow 148 which may be directed to at least one process chamber 154. At least one valve 150 (for example a throttle valve, choker, or similar device) may be positioned between the plasma source 146 and the process chamber 154. In the illustrated embodiment, the valve 150 is to receive the radical gas flow 148 and output at least one valve radical flow 152 to the process chamber 154. Like the previous embodiments, the process gas supply system 142 may be configured to output at least one additional reactant gas 145 which may be directed into the process chamber 154. The process chamber 154 may be configured to permit plasma processing of one or more substrates positioned therein. Further, the process chamber 154 may emit at least one process chamber output flow 156.

Referring again to FIG. 8, at least one gas analyzer sample 160 may be extracted from the valve radical flow 152 and directed to the mass spectrometer 162. The mass spectrometer 162 is configured to output at least one gas analyzer signal 164 to at least one controller 166. In response, the controller 166 may be configured to send and receive data 168 from the valve 150. As such, during use the controller 166 may be configured to regulate the radical flow 148 thereby selectively controlling the pressure of the valve output 152 going into the process chamber 154, which in turn allows selective control of the radical concentration entering into the process chamber 154. Further, the controller 166 may be configured to send control signals 170 to the plasma source 146.

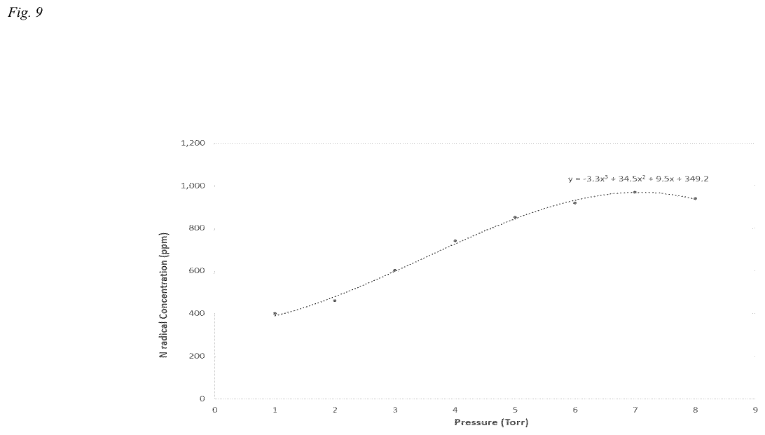

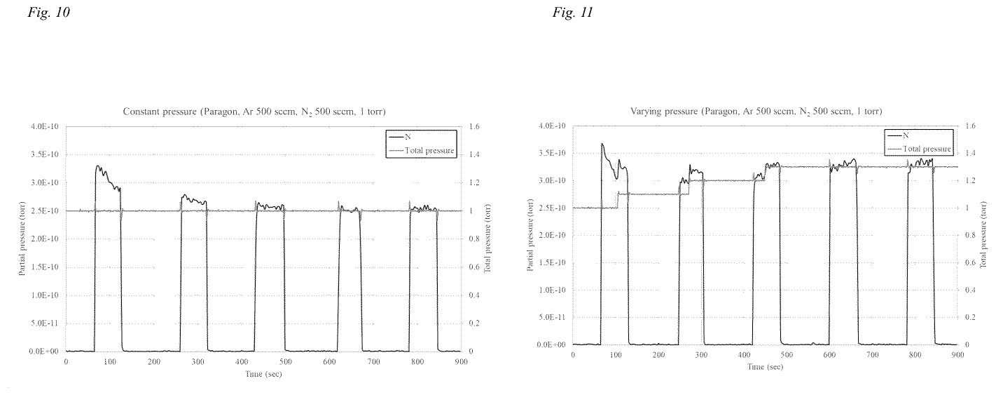

FIGS. 9-11 illustrate graphs showing that effective feedback control for the radical concentration may be achieved by controlling the plasma pressure. More specifically, FIG. 9 shows that a nitrogen radical concentration detected by the mass spectrometer 118 of FIG. 7 may be nonlinear to process chamber 110 pressure. As such, with a fixed process gas flow, adjusting the plasma chamber 110 pressure may achieve the desired radical concentration within a desired range. Similarly, with reference to FIG. 8, adjusting the pressure of the valve output flow 152 entering the process chamber 154 may also achieve the desired radical concentration within the process chamber 154. Those skilled in the art will appreciate that other process gases (e.g. O2, H2, F2, etc.) would yield similar results. FIG. 10 shows that nitrogen radical concentration in the plasma source output would decline in some scenarios over time even the plasma source or chamber pressure remain constant. In contrast, FIG. 11 shows that the nitrogen radical concentration of the plasma source output can be maintained at relatively constant level if the plasma source pressure is varied as compared to FIG. 10.

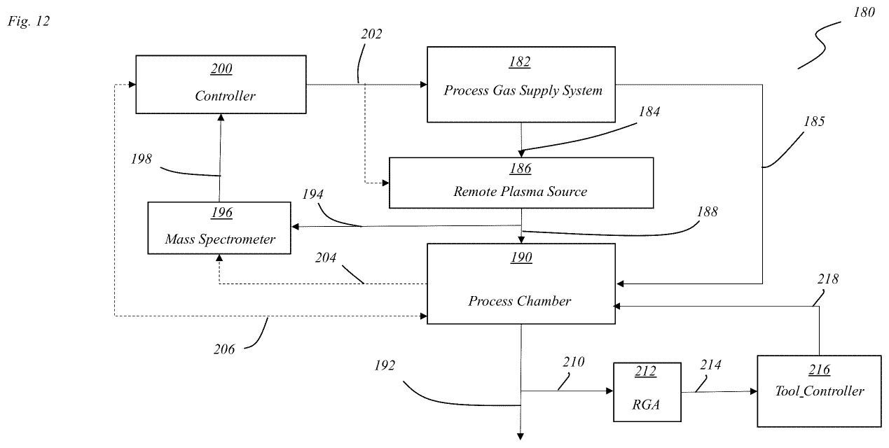

FIG. 12 is a further yet schematic representation of an apparatus having a radical concentration feedback architecture, in accordance with yet another embodiment of the present invention. Like discussed above in connection with the previously described embodiments of the present invention, the apparatus 180 includes at least one process gas supply system 182 configured output at least one process gas 184 to at least one plasma source 186. The plasma source 186 may be configured to output at least one plasma source radical flow 188 which is directed to at least one plasma chamber 190. Further, the process gas supply system 182 may be configured to output and direct at least one additional reactant or precursor flow 185 to the plasma chamber 190. The process chamber may be configured to permit plasma processing of substrates positioned therein. At least one gas analyzer sample 194 may be extracted from the radical flow 188 emitted by the plasma source 186 and directed to the mass spectrometer 196 which in turn generates at least one gas analyzer signal 198. The gas analyzer signal may be directed to at least one controller 200. The controller 200 may send at least one control signal to at least one of the process gas supply system 182 and/or plasma source 186. Optionally, controller 200 may be configured to send at least one control signal to the process chamber 190. The process chamber 186 may be configured to output at least one process chamber output 192. Optionally, at least additional gas analyzer 212 may be configured to examine at least one characteristic of the process chamber output 192. As shown, at least one sample signal 210 may be directed to the additional gas analyzer 212 which in turn generates at least one gas analyzer signal 214. The gas analyzer signal 214 may be directed to at least one tool controller 216. The tool controller 216 may generate at least one tool control signal 218 which is configured to selectively control at least one operational characteristic or parameter of the process chamber 190.

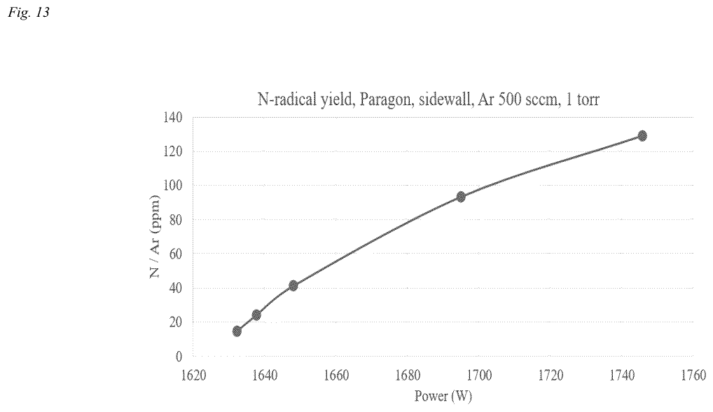

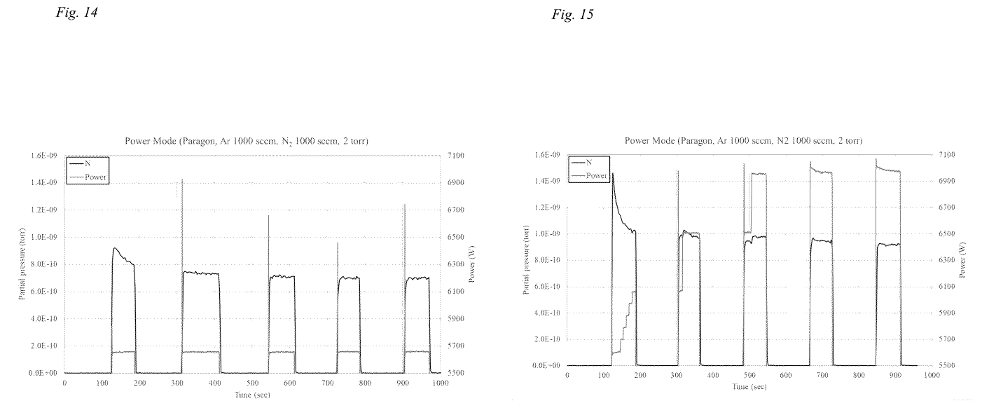

FIGS. 13-15 illustrate graphs showing that effective feedback control of radical concentration may be achieved by controlling the plasma power, in accordance with yet another embodiment of the present invention. FIG. 11 shows that the radical concentration generated by the plasma source 186 illustrated in FIG. 12 is nonlinear to the applied plasma source power. As a result, the radical concentration increases the plasma source power. Therefore, the detected radical concentration (Ci) by the mass spectrometer 196 may be compared to the target radical concentration (Co). For example, if Ci>Co, plasma source power should be reduced. In contrast, if Ci<Co, plasma power should be increased to generate more radicals in the plasma source output 188. FIG. 14 shows the nitrogen radical concentration emitted from the plasma source where constant power is applied to the plasma source. In contrast, FIG. 15 shows the nitrogen radical concentration emitted from the plasma source by applying variable power to the plasma source.

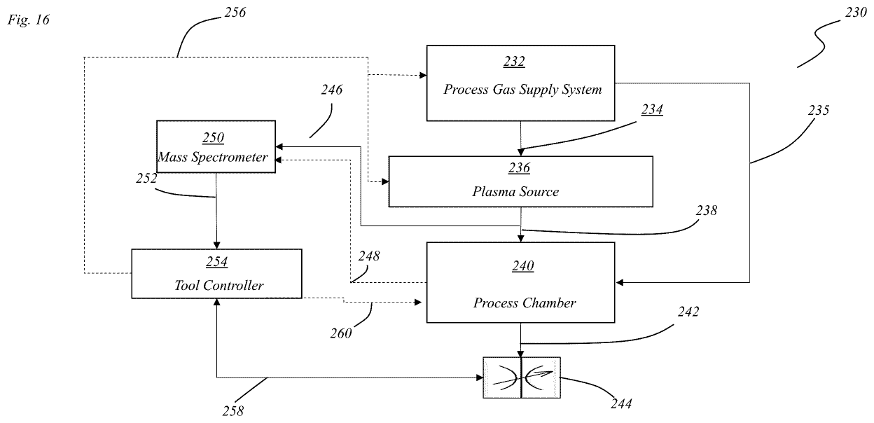

FIG. 16 shows yet another alternate embodiment of a processing apparatus having a radical concentration feedback architecture. As shown, the apparatus 230 includes at least one process gas supply system 232 configured to output at least one process gas 234 to at least one plasma source 236. The plasma source 236 emits at least one radical flow 238 which is directed to at least one process chamber 240. As shown, the process gas supply system 232 may also direct at least one additional reactant or precursor 235 to the process chamber 240. The process chamber 240 may be configured for the plasma processing of one or more wafers, devices, or components positioned therein. Following wafer or device processing, the process chamber 240 emits at least one process chamber output 242. In the illustrated embodiment, at least one valve 244 may be used to control the flow, pressure, and the like of the process chamber output 242.

Referring again to FIG. 16, at least one sample 246 may be extracted from the radical flow emitted from the plasma source 236 and directed to the mass spectrometer 250. Optionally, the mass spectrometer 250 may be configured to receive at least one sample 248 of the radical flow from within the process chamber 240. Thereafter, the mass spectrometer 250 may generate at least one gas analyzer signal 252 which is directed to at least one tool controller 254 based on data from at least one of the radical flow sample 246 or process chamber sample 248. The tool controller 254 may send any number of control signals to any of the components within the system 230. In the illustrated embodiment, the tool controller 254 may direct at least one control signal 256 to at least one of the process gas supply system 232 and the plasma source 236. Optionally, the tool controller 254 may receive data from and provide control signals 258 to the valve 244. Optionally, the tool controller 254 may also send at least one control signal 260 the plasma chamber 240.

The embodiments disclosed herein are illustrative of the principles of the invention. Other modifications may be employed which are within the scope of the invention. Accordingly, the devices disclosed in the present application are not limited to that precisely as shown and described herein.

What is claimed:

1. An apparatus for feedback control in plasma processing systems using radical sensing, comprising:
at least one process gas supply system configured to output at least one process gas;
at least one plasma source configured to receive the at least one process gas and generate at least one radical flow;
at least one process chamber in communication with the at least one plasma source,
wherein the process chamber receives the at least one radical flow and directs at least a portion of the at least one radical flow to one or more devices,
the process chamber configured to output at least one process chamber output;
at least one gas analyzer in communication with and configured to sample at least one of the at least one process gas, at least one radical flow, at least one radical flow within the at least one process chamber, and the at least one process chamber output; and
at least one controller in communication with at least one of the process gas supply system, at least one plasma source, and at least one process chamber,
the controller configured to generate at least one control signal based on data from the at least one gas analyzer and selectively control at least one of the process gas supply system, at least one plasma source, and at least one process chamber.

2. The apparatus of claim 1, wherein, during use, said at least one plasma source is configured to generate low-energy ions and atomic radicals in said at least one radical flow, directed into said one or more process chambers.

3. The apparatus of claim 1, wherein said at least one process chamber is configured to have said one or more substrates or devices positioned therein to be plasma processed.

4. The apparatus of claim 1, wherein said at least one gas analyzer further comprising at least one of a mass spectrometer and a special residual gas analyzer.

5. The apparatus of claim 1, wherein said at least one controller comprising a mass flow controller, and another flow control device.

6. The apparatus of claim 1, wherein said at least one controller, during use, capable of continuously adjusting one or more operational parameters of the apparatus based on gas analyzer data received from the at least one gas analyzer sampling, in real-time, the radical gas flow from said plasma source.

7. A method, comprising:
selecting a reactor configuration;
determining a radical sensing unit to be employed;
determining a reaction rate target;
setting a radical concentration target;
determining a preset flow of other reactants;
flowing one or more process gases into at least one plasma source by initiating at least one plasma reaction;
measuring a radical concentration using the radical sensing unit; and
measuring a reaction rate.

8. The method of claim 7, wherein selecting a reactor configuration comprises selecting of any one or a combination of a plurality of processing gases to be used by said reactor, a plurality of materials to be applied on said reactor, a wafer size to be housed by said reactor, a plurality of dimensions for the reactor, and a type of a remote plasma source for said reactor.

9. The method of claim 7, wherein said radical sensing unit comprising at least one of a mass spectrometry system, and a special residual gas analyzer.

10. The method of claim 7, wherein said reaction rate targets comprising a target deposition rate, a target process time, an etch rate, and a surface modification treatment rate.

11. The method of claim 7, wherein setting a radical concentration target is impacted by at least an initial dose of any gases included in a plasma chamber, and
wherein said dose being dependent upon at least one of disassociation rate by pressure, flow rate, power, and thermal management variables.

12. The method of claim 7, wherein said process gases comprising at least one of O2, N2, H2, NH3, NF3, F2, Cl2, AsH3, BCl3. Br2, CF4, C2F6, C3F8, C4F8, C5F8, CHF3, HBr HCl, HF, N2O, PH3, SiF4, SiH4, SF6.

13. The method of claim 7, wherein said reaction rate may be measured by at least a laser interferometer capable of examining an etch rate, a deposition rate, and a surface modification treatment rate.

14. The method of claim 7, further comprising optimizing a performance of an apparatus for the feedback control by repeating the steps of claim 7.

15. The method of claim 7, further comprising adjusting at least one of or a combination of a gas flow rate, power, cooling characteristics prior to providing the gas for plasma reaction.

17. The method of claim 7, further comprising adjusting at least one of or a combination of a flow rate, and gas mix ratio prior to providing the precursor gas for plasma reaction.
